(19) United States
(12) Patent Application Publication
(10) Pub. No.: US 2006/0211024 A1
(43) Pub. Date: Sep. 21, 2006

(54) Methods for analysis of a nucleic acid sample

(75) Inventors: Robert M. Corn, Corona del Mar, CA (US); Lloyd M. Smith, Madison, WI (US); Timothy G. Burland, Cross Plains, WI (US); Voula Kodoyianni, Madison, WI (US)

Correspondence Address: BOZICEVIC, FIELD & FRANCIS LLP, 1900 UNIVERSITY AVENUE, SUITE 200, EAST PALO ALTO, CA, 94303, US

(73) Assignee: GWC Technologies Incorporated

(21) Appl. No.: 11/372,823

(22) Filed: Mar. 9, 2006

Related U.S. Application Data
(60) Provisional application No. 60/661,286, filed on Mar. 10, 2005.

(51) Int. Cl. C12Q 1/68

(52) U.S. Cl. 435/6

(57) ABSTRACT

An assay for analysis of a nucleic acid sample is provided. In one embodiment, the assay includes: a) employing an RNA sample to obtain a DNA probe; b) contacting the DNA probe with a substrate containing a surface-immobilized RNA oligonucleotide to produce a surface-immobilized RNA/DNA duplex; and c) detecting RNAseH-dependent cleavage of the surface-immobilized RNA oligonucleotide in the RNA/DNA duplex.

### CROSS-REFERENCE TO RELATED APPLICATIONS

This application claims priority benefit of U.S. provisional application Ser. No. 60/661,286, filed Mar. 10, 2005, which application is incorporated by reference herein in its entirety.

### BACKGROUND

In many nucleic acid detection assays, target nucleic acid molecules derived from a sample are contacted with surface-bound polynucleotides under specific binding conditions. Binding of target nucleic acids to the surface-bound polynucleotides can be evaluated to provide an assessment of the abundance of those target nucleic acids in the sample.

This disclosure relates to methods for detecting nucleic acids, e.g., DNA or RNA, in a sample.

### SUMMARY OF THE INVENTION

An assay for analysis of a nucleic acid sample is provided. In one embodiment, the assay includes: a) employing an RNA sample to obtain a DNA probe; b) contacting the DNA probe with a substrate containing a surface-immobilized RNA oligonucleotide to produce a surface-immobilized RNA/DNA duplex; and c) detecting RNAseH-dependent cleavage of the surface-immobilized RNA oligonucleotide in the RNA/DNA duplex.

### BRIEF DESCRIPTION OF THE DRAWINGS

FIG. 1 is a schematic representation of a first embodiment of the instant methods.

FIG. 2A is a schematic representation of a first detection method that may be employed in certain embodiments of the instant methods.

FIG. 2B is a schematic representation of a second detection method that may be employed in certain embodiments of the instant methods. FIG. 3 is a graph illustrating results that would be expected to be obtained by performing angle-scanning prism-coupled SPR for two surface thicknesses (0 nm and 5 nm) of material on the gold layer, indicating how the SPR response changes between the two thicknesses and how SPR response can be used for imaging. FIG. 4 is a schematic representation of an exemplary SPR imager.

FIG. 5 illustrates exemplary results. This figure shows an image of a 25-element SPR array read with an SPR imager. The top row of spots are control spots of surface-immobilized streptavidin pre-blocked with biotin. The 2nd and 3rd rows are control spots of surface-immobilized polyethylene glycol (PEG). The bottom two rows are spots of surface-immobilized streptavidin. The array was exposed to 500 nM biotinylated septathymidine (MW ˜2400). In this figure, the lightness of the spots increases as reflectivity increases when target binds to the spot. Minimal binding to the PEG or pre-blocked streptavidin occurs. These results exemplify how the ability to detect increases or decreases in the mass of molecules attached to the surface can be used to monitor molecular mass on the surface of an array by SPR imaging.

FIG. 6 is a graph of exemplary results. This graph shows a time course of nucleic acid hybridization as measured by SPR imaging. Two 18-mer DNA oligonucleotides of distinct sequence, each with T15 spacers and thiol linkers, were immobilized onto a gold coated SPR substrate. SPR signals in buffer were collected for 2 min, then a 50 nM solution of an 18-mer oligo DNA oligonucleotide complementary to only one of the two immobilized oligonucleotides was introduced into the flow cell and incubated at room temperature. The graph shows the net SPR signal obtained by subtracting the SPR signal for the average of two non-complementary immobilized oligonucleotide spots from the SPR signal for the average of two complementary oligonucleotide spots. Hybridization under these conditions is largely complete within 5-6 minutes.

FIG. 7 illustrates an overview of certain steps employed in certain embodiments of the instant method.

FIG. 8 illustrates prophetic results that would be expected to be obtained by certain embodiments of the instant method. This figure shows oligonucleotide layout (A) and expected SPR signals (B-D) for an SPR array. B: Oligonucleotide array reference image before exposure to sample. C: Expected oligonucleotide array difference image when kanamycin mRNA and not luciferase mRNA is present in the sample. D: Expected oligonucleotide array difference image when kanamycin and luciferase mRNAs are both present in the sample. As would be readily apparent, reflectivity decreases if binding occurs in this assay. In this assay, an increase in binding in this assay therefore produces a spot that is darker.

FIG. 9 shows an exemplary method by which streptavidin (SA) may be linked to an array surface.

FIG. 10 shows an SPR difference image of arrays made using biotinylated and thiolated oligonucleotides.

FIG. 11 shows an SPR image of an array demonstrating that RNaseH specifically degrades RNA in DNA-RNA hybrids. Numbers (mM) indicate the concentration of the oligonucleotide solution spotted. A: Pattern of oligonucleotides spotted on the chip. B: difference image obtained by subtracting reference image from an image taken after array was exposed to 500 nM D1A/R1A DNA complement. C, difference image after array was exposed to 500 nM D2A/R2A DNA complement. D, difference image obtained after further flowing 60 U/mL RNaseH over the chip.

FIG. 12 is a graph showing that an increase in RNaseH concentration causes an increase in the rate of oligonucleotide hydrolysis. An array was fabricated with biotinylated oligonucleotides on a SA surface to assess the effect of RNaseH concentration on oligonucleotide hydrolysis rates at 30° C. At 2500 seconds, 5 nM D2Ac tag was added along with 60 U/ml RNaseH. An image was collected every 3 minutes, the reference image was subtracted and the change in reflectivity for each oligonucleotide was plotted as a function of time There is a clear increase in rate of oligonucleotide removal when the RNaseH concentration is increased.

FIG. 13 is a graph showing that the instant methods can detect 1 fM or less of DNA tag. A an array with biotinylated oligomer oligonucleotides was exposed to increasing concentrations of D1Ac tag (complementary to oligonucleotide RIA) and 60 U/mL RNaseH. Reflectivity changes were calculated from difference images collected at the indicated times and plotted ±SD of the mean of three replicates. At the indicated time, the surface was exposed to the next concentration of tag. Data was normalized for the DNA oligonucleotide complementary to the D1Ac tag.

FIG. 14 is a graph showing RNAse H-catalyzed removal of surface-bound RNA oligonucleotides following tag annealing and separation. Mouse liver mRNA was mixed with luciferase mRNA and DNA tags, heated to 95° C. for 5 minutes, incubated at 50° C. for 25 minutes, then passed over a MicroSpin S-300 gel filtration column and diluted to 600 μL with 120 U/ml RNaseH before exposure to the array. Sample 1: negative control, containing 50 pM each DNA tag with no mRNA, but taken through the annealing and separation procedure. DNA tags are retained in the gel filtration column. Sample 2: a mixture of 1 μg mouse mRNA, 20 ng luciferase mRNA (2% of total mRNA) and 50 pM each DNA tag. The luc DNA tags are recovered in the column eluate. Sample 3: 1 μg mouse liver mRNA, 200 ng luciferase mRNA and 500 pM each DNA tag. Sample 4: 5 nM each DNA tag, added to check the degradability of the oligonucleotide array at the end of the experiment.

FIG. 15 is a graph showing improved sensitivity in detecting specific mRNAs. 1 μg mouse mRNA was mixed with 5 ng luciferase mRNA (˜5% of total mRNA) but no kan mRNA, then mixed with ˜300× molar tag excess of each DNA tag for kanamycin and luciferase mRNAs in 500 mM KCl. The mixture was heated to 95°, gradually cooled to 45°, before it was passed over a MicroSpin S-300 gel filtration column to separate the mRNA-tag hybrids from the unbound tags. RNase H was added to the eluate and the sample was exposed to an array with kan and luc RNA oligonucleotides. SPR shifts monitored on the SPRIMAGER®II were converted to reflectivity changes (D % R). A significant loss of the luc RNA oligonucleotides from the array was observed consistent with detection of luc mRNA. A weak but significant loss of the kan RNA oligonucleotides, consistent with some carryover of control DNA tags was also observed.

FIG. 16 is a graph showing application of the instant method to cDNA targets. 500 ng mouse liver mRNA was mixed with 250 pg luciferase mRNA (˜0.05% of total mRNA). After reverse transcription the mixture was heated to inactivate the enzyme and denature the cDNA, then kept on ice. The sample was diluted, RNaseH was added and the mixture was exposed (see arrow) to an RNA array containing 24-mer RNA oligonucleotides specific for the luciferase mRNA and for the kanamycin control. Loss of signal was observed for both luciferase RNA oligonucleotides, whereas no loss of signal was seen for either kanamycin control RNA oligonucleotides.

### DEFINITIONS

Unless defined otherwise, all technical and scientific terms used herein have the same meaning as commonly understood by one of ordinary skill in the art to which this invention belongs. Still, certain elements are defined below for the sake of clarity and ease of reference.

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length. Oligonucleotides are usually synthetic and, in certain embodiments, are under 80 nucleotides in length, e.g., from about 20 to about 70 nucleotides or about 18 to about 50 nucleotides in length. In certain embodiments, an oligonucleotide may be 20 to 40 nucleotides in length. In other embodiments, an oligonucleotide may be 40 to 70 nucleotides in length.

An “RNA oligonucleotide” is an oligonucleotide that contains at least a region of contiguous ribonucleotide monomers. The ribonucleotide monomer region may bind to a nucleic acid of interest in a sample, and in certain embodiments may be in the range of 20 to 80 monomers in length. In addition to the ribonucleotide monomer region, an RNA oligonucleotide may also contain a region of deoxynucleotide monomers and/or a linker, for example. In one embodiment, an RNA oligonucleotide may be linked to a substrate via a region containing deoxynucleotide monomers.

A “DNA oligonucleotide” is an oligonucleotide that contains at least a portion of deoxynucleotide monomers. The deoxynucleotide monomer portion may bind to a nucleic acid of interest in a sample, and in certain embodiments may be in the range of 20 to 80 monomers in length. In addition to the deoxynucleotide monomer portion, a DNA oligonucleotide may also contain a portion of ribonucleotide monomers and/or a linker. In one embodiment, a DNA oligonucleotide may be linked to a substrate via a region containing ribonucleotide monomers.

The term “RNA sample” as used herein relates to a material or mixture of materials, typically, although not necessarily, in fluid form, e.g., aqueous, containing one or more RNA molecules. Samples may be derived from a variety of sources such as from food stuffs, environmental materials, a biological sample such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). An RNA sample contains RNA molecules. The sample may contain total RNA made from cells (e.g., mammalian or bacterial cells), or any subfraction thereof. For example, an RNA sample may contain isolated mRNA, tRNA, rRNA, unspliced RNA or short RNA molecules (e.g., short interfering RNA (siRNA), micro-RNA (miRNA), tiny non-coding RNA (tncRNA) or small modulatory RNA (smRNA) molecules) that has been isolated from a cell, or made synthetically (e.g., using wholly or partially synthetic (semisynthetic) techniques). Methods for isolating RNA from a cellular sample, as well as methods of making RNA synthetically, are well known in the art. RNA may be isolated from a sample, e.g., an RNA sample by its affinity (e.g., to an oligo-dT oligonucleotides or to gene specific primers), by size, or by any other method.

RNA molecules in a sample may be termed “RNA analytes” herein. In many embodiments, a sample is a complex sample containing at least about 10², 5×10², 10³, 5×10³, 10⁴, 5×10⁴, 10⁵, 5×10⁵, 10⁶, 5×10⁶, 10⁷, 5×10⁷, 10⁸, 10⁹, 10¹⁰, 10¹¹, 10¹² or more species of analyte (e.g., species of RNA). In certain embodiments, a sample may contain a purified analyte.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-immobilized”, with reference to a polynucleotide, refers to a polynucleotide that is (directly or indirectly, covalently or non-covalently) bound to a surface of a solid substrate, where the substrate can have a variety of configurations. In certain embodiments, the polynucleotide is bound to a surface of a planar support.

The term “array” encompasses the term “microarray” and refers to any spatially addressable arrangement of nucleic acid features on a substrate surface.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more than ten thousand features, or even more than one hundred thousand features, in an area of less than 20 cm² or even less than 10 cm², e.g., less than about 5 cm², including less than about 1 cm², less than about 1 mm², e.g., 100μ², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. In certain embodiments, the features of an array may have a diameter in the range of 0.7 mm to 1.0 mm. In one embodiment, the features of an array may have a diameter of at least 50 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm. In embodiments that employ surface plasmon resonance detection, the detected light may have a wavelength in the range of 500 nm to 2000 nm, e.g., 600 nm to 1600 nm or 700 nm to 1250 nm. In particular embodiments, a narrow wavelength or single wavelength of light may be detected.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. Patent Application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. In one embodiment, oligonucleotides may be deposited onto a substrate manually. Inter-feature areas need not be present particularly when the arrays are made by photolithographic methods as described in those patents.

An array is “spatially addressable” when it has multiple regions of different moieties (e.g., different capture agents) such that a region (i.e., a “feature” or “spot” of the array) at a particular predetermined location (i.e., an “address”) on the array will detect a particular sequence. Array features are typically, but need not be, separated by intervening spaces. There may be at least 10-50,000 features on an array (e.g., 50-10,000 or 100-5,000 featues)

In the case of an array in the context of the present application, the “DNA probe” will be referenced as a moiety in a mobile phase (typically fluid), to be detected by “surface-bound” polynucleotides which are bound to the substrate at the various regions. These phrases may be synonymous with the terms “target” and “probe”, or “probe” and “target”, respectively, as they are used in other publications. In certain embodiments, DNA probes may be referred to herein as “DNA tags”.

When an array is “read”, it may be scanned or an image of a region of the array may be produced using a wide-field camera. Depending on the array substrate employed and the methods used, an array may be read by detecting fluorescence on the surface of the array, or by detecting an evanescent wave (e.g., by detection of surface plasmon resonance or the like).

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention, the scan region includes the entire area of the slide scanned (e.g., including, with respect to fluorescent detection embodiments, the area of the slide scanned in each pass of the lens), between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing”, “annealing” and “binding”, with respect to nucleic acids, are used interchangeably.

As will be described in greater detail below, certain embodiments in the instant methods include two hybridization steps: a) one step in which DNA oligonucleotides are hybridized with the RNA molecules of an RNA sample, and b) another step in which a DNA probe is hybridized with the surface-bound RNA oligonucleotides of an RNA array.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determine whether a nucleic acid is specifically hybridized to another nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5× SSC and 0.1× SSC at room temperature.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate. It will also be readily appreciated by the ordinarily skilled artisan that stringent hybridization conditions are selected so as to be compatible with the detection method to be used. For example, where surface plasmon resonance (SPR) is to be used, such SPR assays are often carried out at ambient temperature (e.g., 25-30° C.) and thus compatible hybridization conditions are selected.

The term “specific binding” refers to the ability of a polynucleotide to preferentially bind to a complementary nucleic acid that is present in a mixture of different nucleic acids. Typically, a specific binding interaction will discriminate between nucleic acids in a sample, typically more than about 10 to 100-fold or more (e.g., more than about 1000- or 10,000-fold). Typically, the affinity between two nucleic acids when specifically bound in a duplex (i.e., hybridized to form a hybrid) is characterized by a K_{D} (dissociation constant) of at least 10⁻⁴ M, at least 10⁻⁵ M, at least 10⁻⁶ M, at least 10⁻⁷ M, at least 10⁻⁸ M, at least 10⁻⁹ M, sometimes up to about 10⁻¹⁰ M.

The term “duplex”, or “hybrid” e.g., a “RNA/DNA duplex” or “RNA/DNA hybrid”, is a complex that results from the specific binding of an RNA with an DNA. A RNA and a DNA complementary to the the RNA will usually specifically bind to each other under “conditions suitable for specific binding”, where such conditions are those conditions (in terms of salt concentration, pH, concentration, temperature, etc.) which allow for binding to occur between DNA and RNA to bind in solution. Such conditions, are well known in the art (see, e.g., Ausubel, et al, Short Protocols in Molecular Biology, 5th ed., Wiley & Sons, 2002). “Hybridizing” and “binding”, with respect to polynucleotides, are used interchangeably.

The term “pre-determined” refers to an element whose identity or composition is known prior to its use. An element may be known by name, sequence, molecular weight, its function, or any other attribute or identifier.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not especially distinct. In other words, a mixture is not addressable. To be specific, an array of surface bound polynucleotides, as is commonly known in the art and described below, is not a mixture of capture agents because the species of surface bound polynucleotides are spatially distinct and the array is addressable.

“Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well known in the art and include, for example, ion-exchange chromatography, affinity chromatography, flow sorting, and sedimentation according to density.

The term “assessing” and “evaluating” are used interchangeably to refer to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent.

The term “using” has its conventional, and, as such, means employing, e.g. putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “substrate” is used interchangeably herein with the terms “support” and “solid substrate”, and denotes any solid support suitable for immobilizing one or more oligonucleotides. A “substrate” may contain one or more surface layers, e.g., a self-assembled monolayer.

If one composition is “bound to”, “affixed to” or “immobilized on” another composition, the bond between the compositions do not have to be in direct contact with each other. In other words, bonding may be direct or indirect, and, as such, if two compositions (e.g., a substrate and an RNA oligonucleotide) are bound to each other, there may be at least one other composition (e.g., another layer or a linker) between to those compositions. Binding between any two compositions described herein may be covalent or non-covalent.

A “prism” is a transparent body that is bounded in part by two nonparallel plane faces and is used to refract or disperse a beam of light. The term prism encompasses round, cylindrical-plane lenses (e.g., semicircular cylinders) and a plurality of prisms in contact with each other. In one embodiment, a prism may have a triangular cross-section.

“RNAse H” is any enzyme that cleaves the RNA strand of an RNA/DNA duplex.

If a polynucleotide “corresponds to” or is “for” a certain RNA or DNA, the polynucleotide base pairs with, i.e., specifically hybridizes to, that RNA or DNA. As will be discussed in greater detail below, a particular RNA or DNA and a polynucleotide for that particular RNA or DNA, or complement thereof, usually contain at least one region of contiguous nucleotides that is identical in sequence.

A “label-free” or “unlabeled” polynucleotide is a polynucleotide that is not linked, directly or indirectly, to a detectable moiety, e.g., an optically detectable moiety such as a fluorescent or luminescent label. Label-free detection methods do not include assessing the amount of a label covalently or non-covalently associated with a polynucleotide.

Other definitions of terms may appear below.

### DETAILED DESCRIPTION

A method of sample analysis is provided. In certain embodiments, the method includes: a) contacting an RNA sample with a DNA oligonucleotide under conditions suitable for hybridization of the DNA oligonucleotide to an RNA molecule in the RNA sample to form an RNA/DNA hybrid; b) producing a DNA probe using the DNA oligonucleotide of the RNA/DNA hybrid; c) contacting the DNA probe with a substrate comprising a surface-immobilized RNA oligonucleotide to produce a surface-immobilized RNA/DNA duplex; and d) detecting RNAseH-dependent cleavage of the surface-immobilized RNA oligonucleotide in the surface-immobilized RNA/DNA duplex. Detection of RNAseH-dependent cleavage of the surface-immobilized RNA oligonucleotide indicates the presence of particular RNA molecule in the RNA sample. Detection may done by evanescent wave detection (e.g., by detecting surface plasmon resonance (SPR)), or by detecting a reduction of an optically detectable label linked to the surface-immobilized RNA oligonucleotide (e.g., linked to the distal end of the oligonucleotide, i.e., the end of the oligonucleotide that is not linked to the substrate). Kits are provided for performing the subject method. The subject method finds use in a variety of different applications, including genomics, diagnostic and research applications, e.g., for gene expression assays.

The ordinarily skilled artisan, upon reading the instant specification, will readily appreciate that the method as exemplified herein can be readily adapted for use in other applications. For example, the methods can also be applied to analysis of DNA in a sample. However, for sake of clarity and convenience, exemplary embodiments relating to the analysis of an RNA sample are described herein. Such is not intended to be limiting as to the analyte, but rather only exemplary.

These and other variations will be readily apparent to the ordinarily skilled artisan upon reading the present disclosure.

Before an example of the instant method is described in such detail, however, it is to be understood that method is not limited to particular variations set forth and may, of course, vary. Various changes may be made to the method described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Methods of Sample Analysis

A method of assessing the presence of a nucleic acid of interest in a sample is provided. In an embodiment of particular interest, the nucleic acid is an RNA molecule, e.g., an mRNA, tRNA, rRNA, unspliced RNA or short RNA (e.g., a short interfering RNA (siRNA), a micro-RNA (miRNA), a tiny non-coding RNA (tncRNA) or a small modulatory RNA (smRNA)), in an RNA sample.

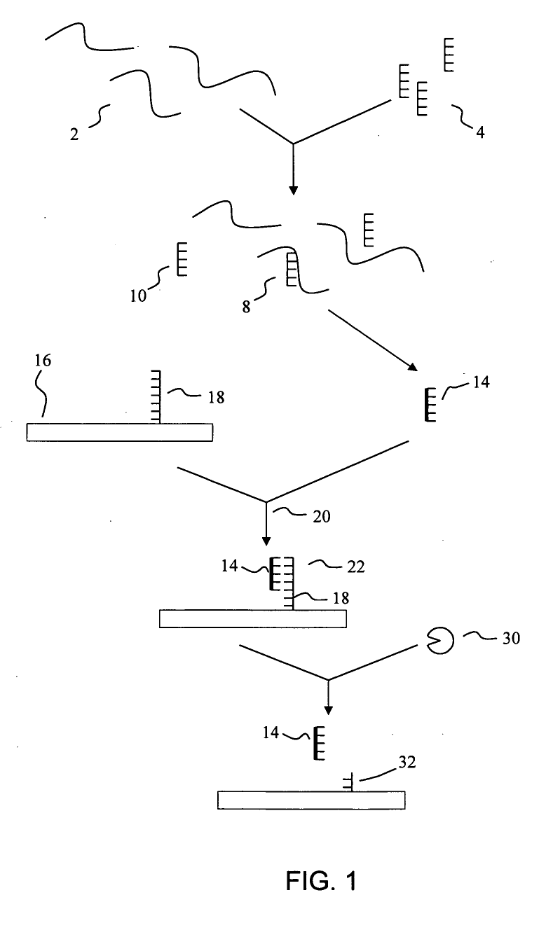

The general features of one embodiment the subject method shown in FIG. 1. With reference to FIG. 1, one embodiment of the subject method includes: a) contacting an RNA sample 2 with a DNA oligonucleotide 4 under conditions suitable for hybridization of the DNA oligonucleotide to an RNA molecule in the RNA sample to form an RNA/DNA hybrid 8; b) using the DNA oligonucleotide in the RNA/DNA hybrid 8 to produce a DNA probe 14, c) contacting the DNA probe 14 with a substrate 16 containing a surface-immobilized RNA oligonucleotide 18 to produce a surface-immobilized RNA/DNA duplex 22 containing the DNA probe 14 and the surface-immobilized RNA oligonucleotide 18 and d) contacting the surface-immobilized RNA/DNA duplex 22 with RNAseH 30 to detectably cleave the surface-immobilized RNA oligonucleotide to produce a cleavage product 32 and release DNA probe 14.

As will be described in greater detail below, the DNA probe may be a DNA oligonucleotide, or an extended product thereof. “Producing a probe” (e.g., as in “producing a DNA probe”) or grammatical equivalents thereof in this context refers to producing a nucleic acid molecule that is subsequently used as a probe due to its ability to form a duplex with a target sequence in a nucleic acid sample. A DNA probe may be produced by, for example, annealing a DNA oligonucleotide to an RNA sample and then extending the DNA oligonucleotide using a polymerase, or by annealing DNA oligonucleotides (which may be referred to as “tags” herein) to an RNA sample and then separating the annealed DNA oligonucleotides from non-annealed DNA oligonucleotides. Both of these methods are discussed in greater detail below. Thus “producing a probe” in the context of this description refers to a probe that is either fully formed prior to sample analysis (as a result of the isolation of annealed oligonucleotides versus non-annealed oligonucleotides), or that is completely or partially formed in vitro during a step of the analysis method (e.g., as a result of nucleic acid polymerization using a target sequence as a template).

Also as will be described in greater detail below, cleavage of the surface-immobilized RNA oligonucleotide may be detected using a variety of means.

As noted above, RNAseH-dependent cleavage of the surface-immobilized RNA oligonucleotide 18 in the surface-immobilized RNA/DNA duplex 18 may be detected using a variety of means, including detection of an optically-detectable label linked to the surface immobilized RNA oligonucleotide 18, or by detection of an evanescent wave (e.g., using a surface plasmon resonance imager). In general terms and in reference to FIG. 1, the presence of the surface immobilized RNA oligonucleotide 18 is detected before, after, and in many embodiments during the period of time in which the surface-immobilized RNA/DNA duplex 22 is contacted with RNAseH 30. In certain embodiments, RNAseH-mediated cleavage of the surface-immobilized RNA oligonucleotide may be detected in real time (i.e., as the cleavage event happens, not afterwards), and may also be detected afterwards.

In certain embodiments, the release of the DNA probe 14 from the surface of the substrate allows the DNA probe 14 to re-hybridize with and facilitate the cleavage of other surface-immobilized RNA oligonucleotides on the surface of the substrate, thereby increasing the sensitivity of the method.

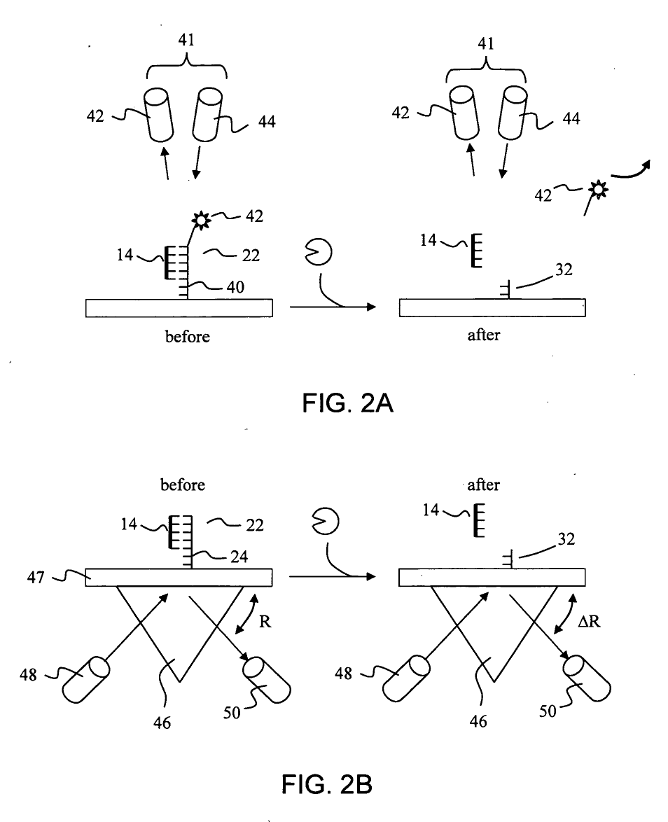

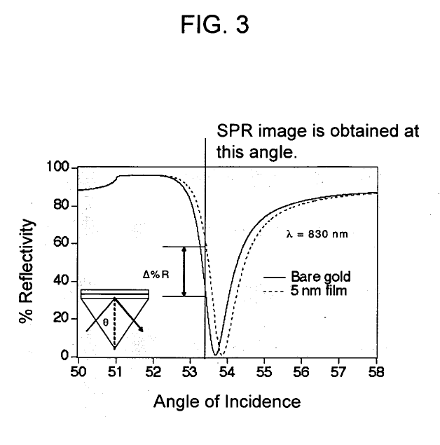

In certain embodiments, cleavage of the surface-immobilized RNA oligonucleotide may be assessed by detecting the presence of an optically detectable label attached to the surface-immobilized RNA oligonucleotide. As illustrated in FIG. 2A, such embodiments generally employ a surface-immobilized RNA oligonucleotide 40 that is linked to (e.g., covalently or non-covalently bound to) an optically detectable label 42 such as a fluorescent, luminescent or colorimetric label. The optically detectable label may be linked to the surface-immobilized RNA oligonucleotide at any point of the nucleotide that will be subject to RNAseH cleavage. As such, the label may be linked to the end of the oligonucleotide that is distal to the end attached to the substrate, or the label may be between the ends of the oligonucleotide. As illustrated in FIG. 2A, signal from the label may be detected and monitored using an optical detection system 41 that generally contains optical detector 42 and optional laser 44.

As illustrated in FIG. 2A, prior to contact with RNAseH, RNA/DNA duplex 22 containing surface-immobilized RNA oligonucleotide 40 linked to label 42 and DNA probe 14 is detectable by virtue of label 42 being present. The presence of label 42 indicates the presence of an intact surface-immobilized RNA oligonucleotide 40. In contrast, after contact with RNAseH and the surface-immobilized RNA oligonucleotide 40 has been cleaved, label 42 is no longer tethered to the substrate surface and is free to move, e.g., diffuse, away from cleavage product 32. Reduced (e.g., little or no) signal from the label is detected in association with the surface immobilized RNA oligonucleotide when the surface-immobilized RNA oligonucleotide is cleaved. It is noted that label may still be detectable after cleavage until such time the label moves, e.g., diffuses, away from the site to which it was attached. Thus, where the method is performed using such a label-based methods, in certain embodiments an end-point measurement may be taken after washing away released labeled entities. Washing may also be employed to remove cleavage enzyme and/or nucleic acid probe (e.g., DNA probe). As previously noted, DNA probe 14 is intact and may travel (e.g., diffuse) and bind other surface-immobilized RNA oligonucleotides and cause their detectable RNAseH-mediated cleavage, increasing the rate of signal loss.

As illustrated in FIG. 2B, cleavage of surface-immobilized RNA oligonucleotide 24 in RNA/DNA duplex 22 may be detected by detecting a change in the amount of reflected light. As will be described in much greater detail in the Examples, such methods generally detect total internal reflection of light at a surface-solution interface that produces an electromagnetic field (an evanescent wave), extending a short distance (typically in the order of less than hundreds of nanometers, e.g., up to about 200 nm) in the z direction (i.e., into the solution), and 20 μm in the x and y directions. These distances may vary greatly depending on the wavelengths of light used. The evanescent wave that occurs outside of a totally internally reflecting prism is sensitive to refractive index changes in the solution in close proximity or in contact with surface of the prism. Binding of DNA probe 14 to surface-immobilized RNA oligonucleotide 24 causes a change in refractive index close to the prism surface. This change in refractive index causes changes in the degree of total internal reflection and the amplitude (and/or, in certain embodiments, the angle) of the reflected light, which can be detected by various means, e.g., by detecting surface plasmon resonance response. In certain embodiments, such evanescent wave detection systems contain a prism 46, an optically matched substrate having a thin metal film at its exposed surface 47, a light emitter 48 and a detector for detecting reflected light 50. Among other methods, in certain embodiments, RNAse-mediated cleavage of the RNA/duplex 22 may be detected by detecting the amplitude and/angle of the reflected light. The angle or intensity of total internal reflectance (R) is increased or decreased when surface-immobilized RNA oligonucleotide 24 is cleaved.

The exemplary method summarized above is described in greater detail below.

As noted above, the DNA probe is produced by contacting an RNA sample with a DNA oligonucleotide under conditions suitable for hybridization of the DNA oligonucleotide to an RNA molecule in the RNA sample to form an RNA/DNA hybrid and producing a DNA probe.

The DNA oligonucleotide employed in the subject methods may contain a nucleotide sequence that allows it to specifically hybridize with a particular RNA molecule that may or may not be in the RNA sample. Accordingly, a DNA oligonucleotide used in the subject methods may specifically hybridize to an RNA (e.g., an mRNA or a miRNA) transcribed from a particular gene, relative to RNAs transcribed from other genes. In certain embodiments, a DNA oligonucleotide employed in the subject methods contains a nucleotide sequence that is complementary to a particular RNA. There is no requirement that an RNA be in an RNA sample for a DNA oligonucleotide corresponding to that RNA to be employed in the instant method. The DNA oligonucleotides employed in these methods are generally unlabeled, i.e., not linked to an optically-detectable moiety.

In particular embodiments, the DNA oligonucleotide employed in the subject methods may be a random primer, e.g., may contain a random nucleotide sequence (i.e., a random sequence of Gs, As, Ts and Cs) and may be in the range of 4-12 nucleotides in length. In certain embodiments, an oligo-dT primer or a gene-specific primer may be employed.

In particular embodiments, the instant methods may employ a mixture of a plurality of DNA oligonucleotides having different nucleotide sequences. The plurality may contain at least 5, at least 10, at least 100, at least 500, at least 1000 or 10,000 or more different DNA oligonucleotides (i.e., oligonucleotides having different sequences). The DNA oligonucleotides in the plurality may hybridize to a plurality of mRNAs transcribed from genes of interest (e.g., genes whose expression is up-regulated or down-regulated in cancer or infected cells and/or control genes) for example. Such DNA oligonucleotides are referred to herein as gene-specific DNA oligonucleotides. In certain embodiments, the plurality of DNA oligonucleotides may contain a plurality of different gene-specific DNA oligonucleotides (e.g., 5-10, for example) that detect an RNA transcribed from a particular gene of interest. In other embodiments, the plurality of DNA oligonucleotides contain random nucleotide sequences. As will be described in greater detail below, such random DNA oligonucleotides may be employed in random priming methods that are well known in the art.

Conditions suitable for hybridization of the DNA oligonucleotide to an RNA molecule in an RNA sample to form an RNA/DNA hybrid are well known in the art, (see, e.g., Ausubel, et al, Short Protocols in Molecular Biology, 5th ed., Wiley & Sons, 2002; Sambrook, et al., and Molecular Cloning: A Laboratory Manual, Third Edition, 2001 Cold Spring Harbor, N.Y.), and readily employed herein. In one representative embodiment, a DNA oligonucleotide may be combined with an RNA sample in water (H₂0), heated to 95° C., for 5 minutes, and cooled to room temperature. Other conditions readily employable in the instant methods may be found in the product literature for reverse transcriptase enzymes (e.g., MMLV or ALV), as sold by a variety of manufacturers such as Stratagene (La Jolla, Calif.), Invitrogen (Carlsbad, Calif.) and Epicentre Inc. (Madison Wis.). In one embodiment, DNA oligonucleotides are annealed to the RNA molecules in an RNA sample in the following conditions: 50 mM Tris, 10 mM MgCl2, and KCl at 100 to 500 mM, depending on stringency wanted, at pH 8. The nucleic acids are annealed at a suitable temperature, e.g., of. 45-50° or 95° and allowed to cool slowly to room temperature.

Referring to FIG. 1, DNA probe 14 may be made from RNA/DNA hybrid 8 using a variety of means. In one example, the DNA oligonucleotide of RNA/DNA hybrid 8 is separated from other DNA oligonucleotides (e.g., DNA oligonucleotide 10) that are not present in an RNA/DNA hybrid. Such separation methods are particularly employed if a plurality of different DNA oligonucleotides are employed in the instant probe-production methods, although such method are readily employed if a single DNA oligonucleotide is used. In one representative embodiment, the hybridized DNA oligonucleotide may be separated from a non-hybridized DNA oligonucleotide by size-exclusion chromatography (i.e., separated by virtue of its being present in an RNA/DNA hybrid that has a significantly higher molecular weight than a non-hybridized DNA oligonucleotide). In another representative embodiment, the hybridized DNA oligonucleotides may be isolated from non-hybridized DNA oligonucleotides by passing the RNA/DNA hybrids and non-hybridized DNA oligonucleotides through an RNA-affinity column, e.g., an oligo-dT column, to isolate the RNA. DNA oligonucleotides hybridized to the RNA co-purify with the RNA. In certain embodiments, a combination of the above size-exclusion and RNA-affinity chromatography methods may be employed. In one embodiment, Straight A's mRNA Isolation System (Novagen, Madison, Wis.) may be used for purification of poly(A)⁺ mRNA from total RNA or tissue lysates. This kit utilizes oligo-dT coupled to magnetic particles that provide an easy way to separate bound oligonucleotides from unbound oligonucleotides. Any chemical agents that are present in the DNA probe after producing the DNA probe, e.g., agents that contain guanidine, may be removed from the DNA probe prior to use.

The DNA oligonucleotides employed herein may be of any suitable size, e.g., from 20-50 nts in length or from 50-80 nts in length, for example.

A hybridized DNA oligonucleotide that has been separated from non-hybridized DNA oligonucleotides may be employed as a DNA probe. Without any covalent modification, e.g., without extension or labeling, the DNA oligonucleotide may be employed as a DNA probe and contacted with a substrate containing a surface-immobilized RNA oligonucleotide.

In another example, a DNA probe may be produced by contacting the RNA/DNA hybrid 8 with an enzyme (e.g., using an RNA-dependent DNA polymerase such as a reverse transcriptase) to extend the hybridized DNA oligonucleotide by primer extension. For example, any one of many suitable reverse transcriptase enzymes (e.g., from MMLV or ALV), as sold by a variety of manufacturers such as Stratagene (La Jolla, CA), Invitrogen (Carlsbad, Calif.) and Epicentre Inc. (Madison Wis.), may be employed in this embodiment. These methods are readily employed with a plurality of DNA oligonucleotides of random nucleotide sequence (i.e., random primers). The primer extension reaction may be terminated at any time to produce DNA primer extension products of any length.

Once produced, a DNA probe may be contacted with a substrate containing a surface-immobilized RNA oligonucleotide without any further covalent modification. In one embodiment, the DNA probe is contacted with an RNA array under conditions that are suitable for RNAseH activity, e.g., 50 mM Tris, 10 mM MgCl2, and KCl at 100 mM, at pH 8, at a temperature of about 30° or 37°, for example. For longer oligonucleotides, a higher hybridization temperature and a thermostable RNaseH (e.g. HYBRIDASE™ from Epicentre) may be employed.

The DNA probe produced by the above exemplary methods may be unlabeled (i.e., the DNA is not labeled by any optically detectable moiety, e.g., a fluorescent, luminescent or dye-containing label) or labeled. In certain embodiments, the DNA probe produced above may be used without any further modification, e.g., without any further extension or labeling thereof. In certain embodiments, the DNA probe may be optionally denatured to release it from the RNA/DNA hybrid, or it may be released from the hybrid using RNAseH, either before or after application of the DNA probe to the immobilized RNA probe. For example, in one embodiment, the DNA probe is released from the RNA/DNA hybrid due to the activity of an enzyme having activity in RNA/DNA-dependent cleavage (e.g., RNAseH), which enzyme can be the same enzyme present in the assay to provide for detection of RNA/DNA duplexes between the DNA probe and the surface-immobilized RNA on the substrate. In such embodiments, the DNA probe is applied to the substrate without the need for further manipulation of the nucleic acid sample to provide for separation of the DNA probe prior to application to the substrate.

As noted above, once produced, the DNA probe 14 is contacted with a substrate 16 containing a surface-immobilized RNA oligonucleotide 18 to produce a surface-immobilized RNA/DNA duplex 22. In certain embodiments, the substrate contains an array of surface-immobilized RNA oligonucleotides. If the DNA probe is produced by the DNA-oligonucleotide separation methods described above, the RNA surface-immobilized oligonucleotide employed in this step of the method is generally complementary to (i.e., will hybridize with) the isolated DNA oligonucleotide. Accordingly, in certain embodiments, if a plurality of DNA gene-specific oligonucleotides are employed for DNA probe production, the array to be contacted with that DNA probe may contain RNA oligonucleotides that are complementary to those DNA oligonucleotides.

A substrate containing a surface-immobilized RNA oligonucleotide, e.g., an RNA array, may be produced using conventional technology. For example, RNA molecules may be synthesized directly on the surface of a substrate using, e.g., nucleoside phosphoramidite chemistry and, in certain embodiments, photolithography. In other embodiments, RNA molecules may be pre-synthesized and attached to the substrate. Depending on the substrate used, a variety of attachment methods may be available. For example, if a substrate is glass, a substrate may be first derivatized by adding reactive silane groups and then linked to an RNA oligonucleotide via a covalent reaction between the reactive silane group and the RNA oligonucleotide. In another embodiment particularly related to surface plasmon resonance applications, the oligonucleotide may be synthesized to have linker having a reactive group (e.g., a thiol group) that can be covalently reactive with the metal, e.g., gold, surface of the substrate, or indirectly via a functional chemical layer that is first attached to the metal. In other embodiments, the metal may be surface modified to provide reactive groups (e.g., thiol groups) that can be reacted with the oligonucleotides. In one embodiment, the metal is surface modified to provide reactive groups that are reacted with streptavidin, and biotinylated oligonucleotides are attached to the streptavidin. Methods for attachment of an oligonucleotide to a substrate for SPR detection are well known in the art (see, e.g., U.S. Pat. No. 5,242,828, describing MUAM layers for oligonucleotide attachment to gold in SPR detection). On one exemplary embodiment, the inter-feature areas of a substrate may be made of a hydrophobic material. The oligonucleotides may be deposited onto the features of such a substrate, and retained therein by the surrounding hydrophobic material.

As employed herein, an array may contain RNA oligonucleotides that are designed to provide specific and strong binding to a target DNA probe. In certain embodiments, the array may contain RNA oligonucleotides that are in the range of 50 to 80 nucleotides in length, although oligonucleotides of a length that is outside of this range (e.g., 20 to 49 or more than 80 nucleotides) are readily employed.

In a particular embodiment described in greater detail below the surface-immobilized RNA oligonucleotide may contain an optically-detectably moiety, e.g., a fluorophore moiety. Specific fluorescent dyes of interest include: xanthene dyes, e.g. fluorescein and rhodamine dyes, such as fluorescein isothiocyanate (FITC), 6-carboxyfluorescein (commonly known by the abbreviations FAM and F),6-carboxy-2′,4′,7′,4,7-hexachlorofluorescein (HEX), 6-carboxy-4′, 5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G⁵ or G⁵), 6-carboxyrhodamine-6G (R6G⁶ or G⁶), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R110, Eosin, JOE, R6G, Tetramethylrhodamine, TAMRA, Lissamine, ROX, Napthofluorescein, Texas Red, Napthofluorescein, Cy3, and Cy5, etc. The optically detectable moiety may be at any position in the surface-immobilized RNA oligonucleotide, e.g. in the middle of the oligonucleotide or at end of the RNA oligonucleotide that is furthest from the substrate.

In a particular embodiment the surface-immobilized RNA oligonucleotide may contain a modifier moiety that is not optically detectable (i.e., is not light or color emitting). In one embodiment, the modifier moiety may be a nanoparticle.

The DNA probe is contacted with the substrate in hybridization buffer, and the DNA probe and surface-immobilized RNA oligonucleotide are allowed to hybridize to produce an RNA/DNA duplex. Again, conditions for nucleic acid hybridization are well known in the art and are readily employed herein (see, e.g., Ausubel, et al, Short Protocols in Molecular Biology, 3rd ed., Wiley & Sons, 1995 Sambrook, et al, Molecular Cloning: A Laboratory Manual, Third Edition, 2001 Cold Spring Harbor, N.Y.)

After the RNA/DNA duplex is produced, RNAseH-dependent cleavage of the surface-immobilized RNA oligonucleotide in the RNA/DNA duplex is detected. In this step, the substrate containing the RNA/DNA duplex is contacted with RNAse (i.e., any enzyme that specifically breaks or hydrolyzes phosphodiester bonds in RNA molecules in an RNA/DNA duplex), and cleavage of the surface-immobilized RNA oligonucleotide in the RNA/DNA duplex is detected.

As noted above, RNAseH-mediated surface-immobilized RNA oligonucleotide cleavage events may be detected using a variety of methods, including by a reduction in signal of a fluorescent moiety linked to the surface-immobilized RNA oligonucleotide, as illustrated in FIG. 2A. Alternatively, as noted above, RNAseH-mediated RNA/DNA duplex events may be detected by monitoring total internal reflectance (e.g., by detecting SPR).

Any cleavage of a surface-immobilized RNA oligonucleotide indicates that an RNA corresponding to that RNA oligonucleotide is present in the RNA sample. The amount or rate of cleavage provides an assessment of the amount of that RNA in the RNA sample.

As noted above, the method as exemplified herein can be readily adapted for use in other applications. For example, the methods can also be applied to analysis of DNA in a sample.

For example, the method can readily be adapted to analysis of DNA in a sample by a) contacting a DNA sample with a DNA oligonucleotide to form DNA/DNA duplexes, wherein a DNA oligonucleotide that is present in such a DNA/DNA duplex is then used to produce DNA probe (e.g., by separating the DNA/DNA duplexes from unduplexed DNA oligonucleotides, then separating the DNA/DNA duplexes into single strands, etc.); b) contacting the DNA probe with a substrate containing a surface-immobilized RNA oligonucleotide to produce a surface-immobilized RNA/DNA duplex; and c) detecting RNAseH-dependent cleavage of the surface-immobilized RNA oligonucleotide in the RNA/DNA duplex. Detection of RNAseH-dependent cleavage of the surface-immobilized RNA oligonucleotide indicates the presence of particular DNA molecule in the DNA sample. Detection may done by evanescent wave detection (e.g., by detecting surface plasmon resonance (SPR)), or by detecting a reduction of an optically detectable label lined to the surface-immobilized RNA oligonucleotide, as described above. The embodiment could be employed in, for example, embodiments in which very large molecules, e.g., an intact eukaryotic (mammalian), bacterial or viral genome or chromosome, is to be investigated using an array.

In further example, the method can be adapted such that the substrate contains a surface-immobilized DNA oligonucleotide, where the method can be adapted to as follows a) contacting a DNA probe (which can be from a DNA sample, generated from a DNA sample, or produced from an RNA sample (e.g., by virtue of production of DNA from an RNA template, by virtue of formation of an RNA/DNA hybrid, etc.) with a substrate containing a surface-immobilized DNA oligonucleotide to produce a surface-immobilized DNA/DNA duplex; and c) detecting DNA/DNA duplex-dependent cleavage of the surface-immobilized DNA oligonucleotide in the DNA/DNA duplex. Detection of DNA/DNA duplex-dependent cleavage of the DNA oligonucleotide indicates the presence of a particular nucleic acid molecule in the sample. Detection may done by evanescent wave detection (e.g., by detecting surface plasmon resonance (SPR)), or by detecting a reduction of an optically detectable label on the RNA contacted with the substrate, as described above.

Utility

The subject methods may be employed in a variety of diagnostic, drug discovery, and research applications that include, but are not limited to, diagnosis or monitoring of a disease or condition (where the expression of a particular RNA is a marker for the disease or condition), discovery of drug targets (where the RNA is differentially expressed in a disease or condition and may be targeted for drug therapy), drug screening (where the effects of a drug are monitored by assessing the level of an RNA), determining drug susceptibility (where drug susceptibility is associated with a particular profile of RNAs) and basic research (where is it desirable to identify the presence of an RNA in a sample, or, in certain embodiments, the relative levels of a particular RNA in two or more samples). In one particular embodiment, the surface bound RNA oligonucleotides may be designed to detect different splice variants, e.g., designed to straddle intron-exon boundaries in a gene or exon-exon boundaries in a cDNA transcribed by the gene. As such, the subject methods may be employed to investigate gene splicing.

In certain embodiments, relative levels of an RNA species in two or more different RNA samples may be obtained using the above methods, and compared. In these embodiments, the results obtained from the above-described methods are usually normalized to the total amount of RNA in the sample or to control RNAs (e.g., mRNAs expressed by constitutively expressed genes), and compared. This may be done by comparing ratios, or by any other means. In particular embodiments, the RNA profiles of two or more different samples may be compared to identify RNA that are associated with a particular disease or condition (e.g., an RNA that that is induced by the disease or condition and therefore may be part of a signal transduction pathway implicated in that disease or condition).

The different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells. The subject methods are particularly employable in methods of detecting the phosphorylation status of phosphorylated serum proteins.

As would be readily apparent, the above described methods are highly sensitive and, as such, may in certain embodiments may be employed in protocols in which nucleic acid amplification methods (e.g., PCR or T7 polymerase-based amplification methods) are not employed. In particular embodiments, e.g., particularly in embodiments that employ unlabelled DNA probes, certain problems (e.g., signal bias etc.) that can be present in label-based detection methods, are avoided.

Accordingly, among other things, the instant methods may be used to link the expression of certain genes to certain physiological events.

Kits

Also provided herein are kits for practicing the subject methods, as described above. The subject kits contain at least an unlabeled DNA oligonucleotide; and RNAseH, as described above. The kit may also contain reagents for preparing an RNA sample, reagents for hybridizing DNA oligonucleotide to RNA in an RNA sample, reagents for separating hybridized from non-hybridized DNA oligonucleotides, control RNA oligonucleotides, control DNA oligonucleotides, or a buffer for surface immobilizing labeled or unlabeled RNA oligonucleotides, etc. The unlabeled DNA oligonucleotide may contain 4 to 12 nucleotides of random nucleotide sequence, or may specifically hybridize to a particular species of mRNA. The various components of the kit may be present in separate containers or certain compatible components may be precombined into a single container, as desired.

In addition to above-mentioned components, the subject kits may further include instructions for using the components of the kit to practice the subject methods, i.e., to instructions for sample analysis. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Surface Plasmon Resonance Detection

Surface Plasmon Resonance (SPR) is based upon the generation of surface plasmons (SPs), which are surface electromagnetic waves that propagate parallel to a metal/dielectric interface. SPs are created when the energy from p-polarized incident photons is coupled into oscillating modes of free electron density present in the metal film. SPs are evanescent waves that have the maximum charge density at the interface and decay exponentially away from the phase boundary to a penetration depth on the order of 200 nm. SPs cannot be excited directly at a planar air/metal or water/metal interfaces because momentum-matching conditions cannot be satisfied. Therefore, it is necessary to use a prism or a grating coupling arrangement to excite SPs. SPR systems may use attenuated total internal reflection prism coupling in the Kretchmann configuration (Homola et al., Sensors and Actuators 1999 B 54, 16-24). Here a thin metal layer (˜50 nm) is placed in direct optical contact with a prism (FIG. 3). In this arrangement, the evanescent light wave produced at the prism/metal interface during total internal reflection excites the SP modes. Typically, SPR measurements are collected in one of three modes, angle shift, wavelength shift or SPR imaging, which uses a fixed angle and fixed wavelength.

Angle shift measurement employs a single wavelength for excitation and measures the reflectivity from the prism/gold/film assembly as a function of incident angle. As shown in FIG. 3, as the incident angle is increased the reflected intensity is damped, due to the creation of SPs. The minimum in the reflected intensity is known as the SPR angle. Both the position of the SPR angle and the resonance curve shape are very sensitive to changes in the index of refraction and the thickness of the film at the metal surface. FIG. 3 shows the shift in the SPR curve (dotted line) upon adsorption of a 5 nm film. Quantitative measurements of the SPR angle shift are the basis of scanning angle detectors.

An alternative SPR measurement approach is to maintain a fixed incident angle and measure reflectivity as a function of wavelength. In this case, a minimum in reflectivity occurs at a specific wavelength, and the position of this minimum shifts upon absorption of material at the interface. Recent improvements in these types of measurement (Frutos et al., Anal. Chem. 2001 71, 3935-3940), using the light source from a Fourier transform (FT) infrared spectrometer, form the basis of certain detection systems. These systems are highly sensitive.

In certain SPR methods, both the angle of incidence and wavelength of light are fixed. Spatial differences in reflectivity due to differences in film thickness or index of refraction are measured across the sensor surface. Capturing the SPR signal at the same angle of incidence (FIG. 3, vertical line) before and after target binding to surface bound molecules generates a reflectivity difference, Δ% R. For an appropriate angle of incidence, the SPR signal is linear with respect to surface coverage provided Δ% R≦10% (Nelson et al., Anal. Chem. 2001 73, 1-7). SPR imaging is thus most simply performed by taking two measurements: one before binding and one after. The pre-binding signals are then subtracted from the post-binding signals to generate a difference image. The presence of control surface-bound molecules exposed to the target at the same time as the experimental molecules on the same array, gives the SPR imaging method inherent controls that other SPR methods lack.

### Example 2

### Surface Plasmon Resonance Imaging Systems

An exemplary SPR imaging system is illustrated in FIG. 4. Light from a collimated polychromatic source passes through a polarizer and impinges on a sample cell at a specific angle near the SPR angle (the sample cell consists of a prism, a glass chip coated with a thin layer of gold to which the oligonucleotides are attached, and a flow cell that keeps the target molecules in contact with the oligonucleotides). A pump moves the sample from a reservoir into the flow cell. A rotation stage positions the sample cell at the appropriate angle in the path of the polarized light. The light interacts with the prism-gold interface, generating surface plasmons (SPs) which in turn attenuate the light reflected from the surface. The light reflected from the sample then passes through a narrow bandwidth filter and on to the detector.

The detector in this imager is a CCD camera, which captures an image of the entire optical field of the substrate surface. Two-dimensional imaging is done by focusing the reflected light with an imaging quality lens onto the camera. The images are monitored in real-time with standard frame capture and image processing techniques. Images are then analyzed using image analysis software to produce data. The collected data allow (i) the analysis of the oligonucleotide regions referenced to images acquired at other times in the experiment and (ii) the measurement of intensity differences between different oligonucleotide-containing regions on the same image. Simultaneous monitoring of the entire array surface facilitates robust experimental controls that guard against perturbations that might be caused by slight changes in temperature or buffer refractive index. And, changes in reflectivity can be monitored and viewed in real time, as they occur. This system is used routinely to monitor arrays of up to 96 features, though the system is capable of imaging many more features without modification. This system makes use of a CCD camera and white light.

In use, an array is mounted on the sample cell, buffer is allowed to flow into the flow cell, and a reference image of the array is collected. The sample containing unlabelled target molecules that bind the oligonucleotides on the surface of the array is then allowed to flow over the substrate. After incubation, a post binding image is collected. Subtracting the reference image from the post binding image creates the difference image.

### Example 3

### Detection of RNAse mediated cleavage

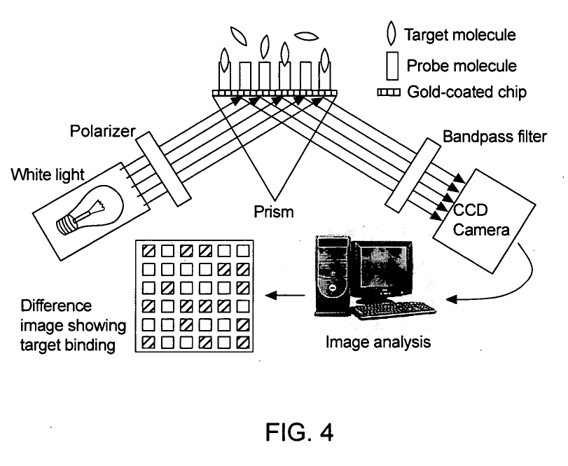

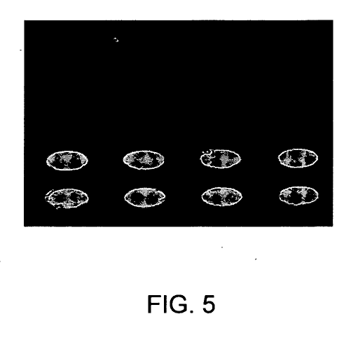

To illustrate that the method is functional, an experiment was designed using this fabrication method to illustrate performance. Results are shown in FIG. 5. The results indicate that using this fabrication method, binding of biotinylated-T₇ (a˜2400-dalton target molecule) to surface-immobilized streptavidin is readily detected by SPR imaging.

FIG. 5 demonstrates that adsorption as well as loss of material from the substrate surface can be readily measured, as well as that biotinylated oligonucleotides may be employed.

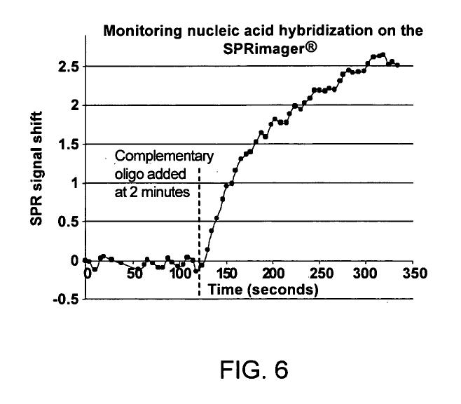

This system may be used to detect gene expression by monitoring nucleic acid hybridization over time, rather than via end-point measurements alone. To show that the SPRimager can be used to collect such data in real time, a DNA-DNA hybridization experiment was performed by SPR imaging (FIG. 6). The time course of hybridization of DNA oligonucleotides to complementary surface-immobilized DNA oligonucleotides was readily measured, therefore the time course of the loss of nucleic acid material from the surface can likewise be readily measured (this is stated later).

When the DNA probes hybridize to the RNA oligonucleotide, the RNA can be degraded by RNAseH (RNase H degrades only the RNA strand in RNA-DNA heteroduplexes). This removes the probe from the biochip surface and releases the target DNA, which is then able to bind to another cognate RNA oligonucleotide on the array, whereupon the RNAseH again degrades the oligonucleotide. One target DNA molecule can degrade up to 12,000 or more RNA oligonucleotides in consecutive rounds (J. Am. Chem. Soc. 2004 126 4086-4087). The amplification is linear rather than exponential, which may help to avoid quantification artifacts.

### Example 4

### Experimental Design

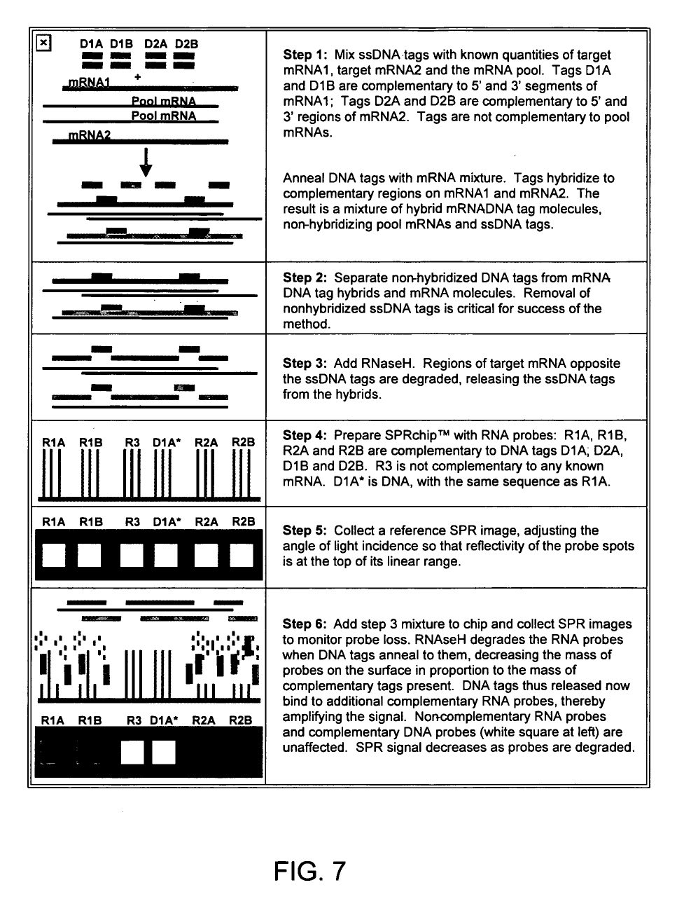

FIG. 7 is an overview of one strategy that may be employed. The aim is to to detect specific mRNAs in a complex mix of mRNAs, by hybridizing ssDNA oligomers (tags) to the mRNAs, separating the annealed tags from the non-hybridizing tags, then exposing an RNA array to the ssDNA tag/mRNA mixture. The tags recovered with their complementary mRNAs reflect the abundance of the specific mRNA in the mixture. On-chip amplification will occur when RNase H then (i) releases the tags from their target mRNAs, making them available for hybridization to the RNA oligonucleotides on the SPRchip and (ii) hydrolyzes the RNA oligonucleotides in RNA-DNA hybrids thus making the same tag available to anneal to another RNA oligonucleotide molecule.

The purpose of the experiment is to monitor the levels of mRNA molecules whose mass ratio in a total cellular mRNA mixture is known with confidence. Human liver poly(A) mRNA (Ambion, cat#7961) is used as the RNA sample. Two control non-human mRNAs are used to estimate feasibility and sensitivity of detection: mRNA1 will be a 1.2 kb kanamycin (kan) RNA (Promega, cat #C1381) and mRNA2 will be a 1.8 kb luciferase (luc) RNA (Promega cat#L4561). This combination of target and pool RNAs is chosen to ensure that the background of mRNA in the pool with complementarity to the target mRNAs is as close to zero as possible.

The following oligonucleotides are employed:

Location
on Gene

| | | | | | | | |
|---|---|---|---|---|---|---|---|
| Tag | Key | Sequence | Bases | Lend | Rend | Tm | ΔG |

| | | | | | | | |
|---|---|---|---|---|---|---|---|
| kanL | D1A | GGATAAAATGCTTG | 24 | 357 | 380 | 58.0 | −45.9 |
| | | ATGGTCGGAA | | | | | |
| kanR | D1B | ATCCTCTAGAGTCG | 24 | 1124 | 1147 | 60.0 | −45.9 |
| | | CCACGGTTGA | | | | | |
| lucL | D2A | CTCTCCAGCGGTTC | 24 | 96 | 119 | 59.3 | −45.9 |
| | | CATCCTCTAG | | | | | |
| lucR | D2B | TTTTCGCGGTTGTT | 24 | 1555 | 1578 | 59.1 | −46.4 |
| | | ACTTGACTGG | | | | | |
| caoB | R3 | GGAUGGGAAUACUC | 24 | 925 | 948 | 59.8 | −46.0 |
| | | AACCGAUGGA | | | | | |

These DNA oligonucleotides are chosen using PrimerSelect software v5.08 from DNASTAR's Lasergene suite using the following criteria: a) they are 24 residues long, and one is from the 5′ third and one is from the 3′ third of each gene (except only one probe is needed for the R3 control); b) they have well-matched ΔG and melting temperature (Tm) to facilitate similar annealing profiles; c) there is limited or no complementarity between the oligos; and d) there are no internal complementarities that might encourage hairpin formation.

The DNA oligos were then checked for limited complementarity with human genes. Each oligo was used as a query to search the human genome NR and human EST databases using NCBI BLAST for short queries. Maximum matches tolerated were 17/24 nucleotides.

The corresponding RNA oligonucleotides immobilized on the SPR chips consist of sequences complementary to these four DNA oligos with 5′ thiol plus spacer modification. The purpose of the spacer is to raise the RNA oligonucleotides off the surface and make them more available to the DNA probes. The thiol is the functional group for surface attachment.

For example, the sequence of the RIA oligonucleotide ready for attachment to the gold surface is:
R1A: HS—(CH₂)₆—(A)₈—UUCCGACCAUCAAGCAUUUUAUCC

In addition two control RNA oligonucleotides are used. R3, a segment of the Arabidopsis thaliana cytochrome synthase B gene, is an RNA oligonucleotide that is not recognized by any DNA oligonucleotide used and has limited complementarity to entries in human NR and EST databases (RNA negative control). D1A* has the complementary sequence to DIA, but is a DNA oligonucleotide rather than an RNA oligonucleotide. No significant signal change for D1A* is detected since RNase H does not cause significant or any detectable degradation of the D1A/D1A* dsDNA.

RNA and DNA oligonucleotides are purchased with a 5′thiol-modification and (CH₂)₆-A6 spacers from Dharmacon RNA Technologies (Lafayette, Colo.) or Integrated DNA Technologies (IDT, Coralville, Iowa). The Dharmacon oligonucleotides are deprotected and HPLC purified by the manufacturer and used as received.

Features having covalently linked polyethylene glycol (PEG) are also employed as controls for system stability. Such PEG surfaces are resistant to nonspecific adsorption (FIG. 5).

### Example 5

### Annealing DNA Oligonucleotides to mRNAs

One microgram of mouse mRNA is mixed with varying quantities of kan mRNA, luc mRNA and the DNA oligonucleotides. All steps involving RNA are performed under conditions that avoid and/or inactivate RNases; all buffers are treated with DEPC and autoclaved. Whenever possible RNase free materials are used.

The mRNA/DNA oligonucleotide mixture is heated briefly to 90° C. to denature any secondary structures, then the mixture is allowed to come to room temperature over a period of one hour to allow the DNA oligonucleotides to anneal to their corresponding mRNAs. Different salt concentrations are tested to identify conditions that minimize or completely remove non-specific annealing of the DNA oligonucleotides to mRNAs.

### Example 6

### Removal of Non-Hybridizing ssDNA Tags

DNA oligonucleotides that are hybridized to mRNA molecules (i.e., hybridized DNA oligonucleotides) are separated from DNA oligonucleotides that are not hybridized to mRNA molecules (i.e., non-hybridized DNA oligonucleotides). Carryover of non-hybridized DNA oligonucleotides may be detected by using controls, e.g., by mixing mouse mRNA and kan mRNA, but not luc mRNA. Any loss of signal for the control RNA oligonucleotides on the array indicates carryover of non-hybridized DNA oligonucleotides.

The RNeasy MinElute Cleanup Kit (QIAgen cat# 74204), designed to purify RNA using a silica-gel-membrane. Only RNA molecules >200 bp long are isolated so free DNA oligonucleotides are removed. According to the manufacturer, picogram quantities of RNA can thus be concentrated.

Two or more rounds of separation may be employed.

The purified product contains mRNA1 and mRNA2 with the DNA oligonucleotides annealed to them, plus pool mRNAs with no DNA oligonucleotides hybridized and no free DNA oligonucleotides. Due to the large molar excess of tags, there are negligible levels of mRNA1 and mRNA2 with no DNA oligonucleotides annealed to them.

### Example 7

### Addition of RNAseH

Ribonuclease H (RNase H) is an endoribonuclease that specifically hydrolyzes the phosphodiester bonds of the RNA strand in an RNA-DNA hybrid, to produce 5′phosphate teminated, 3′-OH products and single stranded DNA. RNase H does not hydrolyze ssDNA, ssRNA, dsDNA or dsRNA. RNase H (Takara Mirus Bio., cat#TAK1250C) is added to the mRNA/DNA oligonuceotide mixture from example 6 above at 1-120 units/ml. The RNAseH concentration employed may be optimized.

### Example 8

### Step 4: Array Fabrication

RNA oligonucleotides are attached to the gold surface of the SPR substrate using well established attachment chemistry (Brockman et al, J. Am. Chem. Soc. 1999 121, 8044-8051). Briefly, the gold-coated glass chip is first immersed in a 1 mM ethanolic solution of MUAM (11-mercapto-undecylamine) to create a self-assembled monolayer (SAM) with an exposed amine functional group. Addition of 1 mM solution of the hetero-bifunctional linker SSMCC (sulfosuccinimidyl4-(N-maleimido-methyl)cyclohexane-1-carboxylate) results in a thiol-reactive, maleimide-terminated surface. RNA and DNA oligonucleotides with a 5′thiol modification are spotted on this surface to produce an array with covalently attached, oriented oligonucleotides. Oligonucleotide attachment following this protocol with the A₆ spacer may facilitate better interaction with the target molecules than does direct attachment of the thiol-DNA to the gold (Nelson et al, Anal. Chem., 2001 73, 1-7). PEG (MW2000) is immobilized on control spots using PEG-NHS ester (NekTar). PEG has been shown to have low non-specific affinity for nucleic acids polymers.

The SPR imager system used need not contain the dextran matrix used for other SPR measurements, so an attachment chemistries that offer good control over oligonucleotide density and availability may be used. Oligonucleotide attachment following the proposed protocol results in ˜1×10¹² molecules cm⁻² (Nelson et al, Anal. Chem. 2001, 73, 1-7), so oligonucleotide concentration on the chip surface is adequate to generate a wide dynamic range of SPR signal (reflecting a theoretical maximum fall from 10¹² to zero molecules cm⁻²) for monitoring loss of RNA oligonucleotides.

Another benefit of the use of SPR imaging over methods that employ labeled probes is that the quality of the oligonucleotide array may be determined before exposure of the array to the experimental sample. Since the reflected light changes proportionally to the thickness of the layer attached to the gold, array areas that carry oligonucleotides appear lighter on a darker background. Therefore array quality and uniformity will be readily assessed when taking the reference image at the commencement of the SPR analysis.

Arrays may be manually or mechanically spotted.

### Example 9

### Analysis of the Reference SPR Image

Immediately after fabrication, the array is mounted on the SPR imager with the flow cell filled with buffer only. The angle of the incident light is adjusted to the optimum for sensitivity and linearity of the SPR curve (FIG. 3).

Typically, for each time point, multiple images are collected and averaged. The more images that are averaged, the lower the system noise and greater the sensitivity of the measurement. For initial experiments at least 5 images are averaged per data point. However, the number of images per data point may be increased as necessary to achieve maximum system performance. 30-100 images may be averaged per data point. The reference image (also an averaged image) will be subtracted from each averaged image to determine the SPR responses to changes induced by the RNase H mixture.

### Example 10

### Data Collection and Analysis

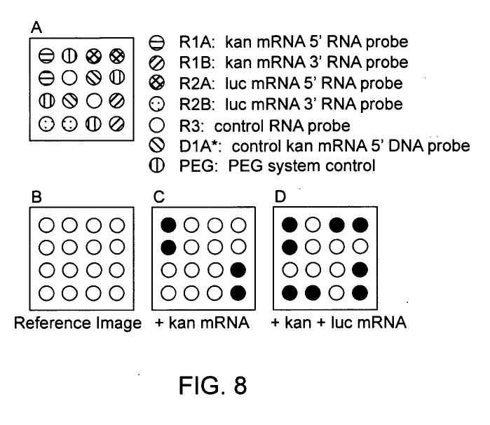

Once the reference image is acquired (FIG. 8), the mixture of RNAseH, mRNAs and mRNA/DNA oligonucleotide hybrids are introduced into the flow cell, and images (data points) will be collected after 5, 10, 20, 60, 120 and 240 minutes.

Images can be collected in continuous mode with a programmed delay between averaged images. Images collected at each time point will be saved so that “difference images” can be obtained. In addition, numerical data are saved to a spreadsheet and a chart is created in real time. Each data point charted is the averaged reflectivity measurement of each oligonucleotide spot after subtraction of the reflectivity of the corresponding region of the reference image. The rate of change in reflectivity for the experimental spots reflects the abundance of the corresponding RNA in the sample. The reflectivity of control spots remain substantially unchanged. In the event control spot reflectivities do change, for example due to system drift or impure reagents, changes in control spots can be used to correct reflectivities of experimental samples for these effects.

The above RNAseH-based method is applicable to both label-free and fluorescent labelling approaches. For label-free detection by SPR, the loss of mass from the surface of the biochip (FIGS. 7 and 8) is just as readily measured as signal gain on an SPR imager. For fluorescence detection, the immobilized RNA oligonucleotides would be labeled (rather than the more typical approach of labeling the DNA molecules in solution), and loss rather than gain of fluorescence would be measured.

All SPR images are collected and analyzed using a combination of commercial and other software. Signals are corrected by subtracting the signal for system control, the PEG signal. Any significant deviations in the signals from the two other controls may be examined to determine the cause, and solutions will be explored.

Current gene expression microarray systems have a small dynamic range: high abundance mRNAs become saturated while low abundance mRNA signals become lost. The SPR detection methods described above offers a solution to this problem: real time data collection can allow the rate of signal change to be monitored. The rate of signal loss reflects the concentration of DNA oligonucleotides in the DNA probe contacted with the array which in turn reflects the abundance of the complementary mRNA species the DNA oligonucleotides anneal to. Taking measurements over a time course extends the dynamic range over which mRNA abundances can be measured. In other words, the rate of signal loss may be used to calculate mRNA abundance. Time course measurements are particularly applicable to non-labeled implementations such as SPR imaging since the problem of quenching of e.g. fluorescent labels by exciting wavelengths is eliminated.

### Example 11

### Array Fabrication and Oligonucleotide Design

RNA arrays on a gold surfaces were fabricated using well developed methods developed for DNA arrays on gold surfaces. For example, RNA oligonucleotides were synthesized with terminal thiol groups that can then be immobilized on maleimide-activated gold surfaces (e.g. Goodrich et al, J. Am. Chem. Soc. 2004 126 4086-4087;

Goodrich, et al. Anal. Chem. 2004 76, 6173-6178), or by attaching biotinylated RNA oligonucleotides to a streptavidin-modified gold surface.

SPOTREADY™ arrays (GWC Technologies, Madison, Wis.) were employed as substrates. These 18 mm×18 mm chips, designed for GWC's SPRIMAGER®II, contain gold spots on a hydrophobic background. The arrays were spotted manually using a micropipet. The array contained negative control oligonucleotides that were exposed to the target (analyte) at the same time under the same conditions as the experimental oligonucleotides.

Oligomers and Probe Design

The methods described in this example require oligonucleotides at two stages: (i) “DNA Tag” oligos that are complementary to specific mRNA targets and are hybridized to mRNA populations to generate mRNA-tag hybrids that are then purified from unhybridized tags; and (ii) RNA oligonucleotides that are complementary to the specific DNA tags and immobilized on arrays suitable for hybridizing to the purified DNA thereby making DNA-RNA hybrids that are susceptible to degradation by RNAseH.

Oligomer DNA tags containing 24 residues complementary to kanamycin and luciferase mRNAs and RNA oligonucleotides complementary to the DNA tags were designed using PrimerSelect software v5.08 from the Lasergene suite (DNASTAR, Madison, Wis.), applying the following criteria: well-matched ΔG and melting temperature to facilitate annealing under similar conditions, minimal complementarity between the oligos or with mouse gene mRNAs and minimal internal complementarities that might encourage hairpin formation.

Control DNA probes had the same sequence as the corresponding RNA oligonucleotides (e.g. DIA and RIA), whereas DNA tags have sequence complementary to the RNA oligonucleotides (Table 1. R3 is a negative control RNA oligonucleotide from the Arabidopsis thaliana cytochrome synthase B gene that is not expected to be recognized by any tag and therefore not degraded in the assay. The A₈ spacer is designed to raise the oligonucleotides off the attachment surface, allowing them to be more accessible to target DNAs.

TABLE 1

Sequences of oligonucleotides and tags used

| | | |
|---|---|---|
| DNA TAGS | KEY | SEQUENCE |

| | | |
|---|---|---|
| kanL | D1Ac | GGATAAAATGCTTGATGGTCGGAA |
| kanR | D1Bc | ATCCTCTAGAGTCGCCACGGTTGA |
| lucL | D2Ac | CTCTCCAGCGGTTCCATCCTCTAG |
| lucR | D2Bc | TTTTCGCGGTTGTTACTTGACTGG |
| caoB | D3c | GGAUGGGAAUACUCAACCGAUGGA |

| | | |
|---|---|---|
| Oligos | KEY | SEQUENCE |

| | | |
|---|---|---|
| Thiol- | Thiol- | HS-(CH₂)₆-AAAAAAAAUUCCGACCAUCAAGCAUU |
| kanL | R1A | UUAUCC |
| Biotin- | Biotin | Biotin-AAAAAAAAUUCCGACCAUCAAGCAUUUUA |
| kanL | R1A | UCC |
| kanL-DNA | D1A | Biotin-AAAAAAAATTCCGACCATCAAGCATTTTA |
| control | | TCC |
| Biotinl- | R2A | Biotin-AAAAAAAACUAGAGGAUGGAACCGCUGGA |
| lucL | | GAG |
| Biotin- | R2B | Biotin-AAAAAAAACCAGUCAAGUAACAACCGCGA |
| lucR | | AAA |
| Biotin- | R3 | Biotin-AAAAAAAAUCCAUCGGUUGAGUAUUCCCA |
| caoB | | UCC |

In some experiments PEG (polyethylene glycol) was used as a further control spot that is expected to remain constant during the experiment.

Immobilization of biotinylated oligonucleotides: 5′ Biotin-conjugated DNA and RNA oligomers were obtained from IDT (Integrated DNA Technologies, Coralville, Iowa) and were used for chip fabrication as received without further purification.

Chip Fabrication

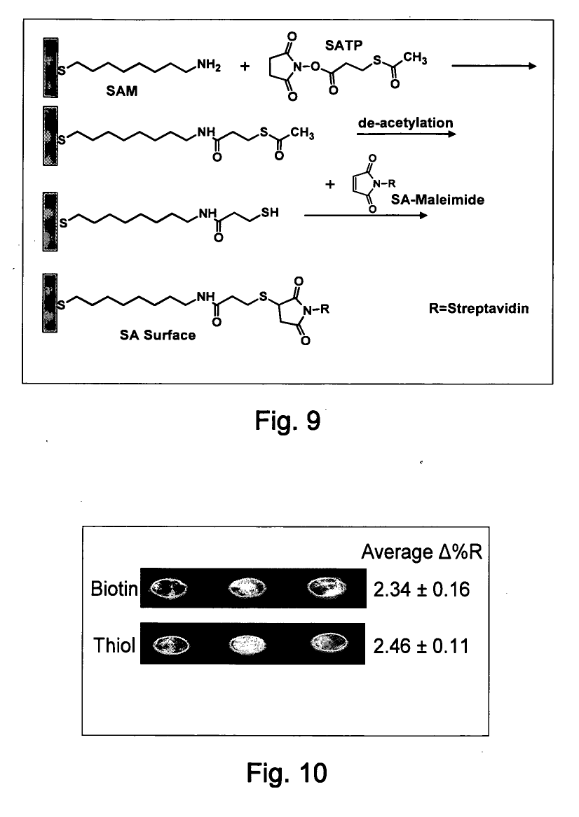

Streptavidin (SA) surfaces on SPOTREADY™ chips were fabricated using the scheme shown in FIG. 9. Briefly, chips were incubated in 1 mM AOT (8-amino-octanethiol, Dojindo Molecular Technologies, Gaithersburg, Md.) to generate an amine-activated surface. After washing with ethanol, the surface was reacted with SATP (N-succinimidyl S-acetylthiopropionate, PierceBio, Rockford, Ill.) and then de-protected with hydroxylamine to generate free thiols. An excess of streptavidin-maleimide (140 μM) was covalently attached to the thiol groups. Biotinylated oligonucleotides were spotted on the SA surface and allowed to bind for 15 minutes before the array was rinsed with PBS and mounted on the SPRIMAGER®II. SPR signals were collected after hybridization of complementary tag DNA to the array, and were then converted to Δ% R, an absolute measure of the change in reflectivity.

Immobilization of thiolated oligonucleotides: Thiolated RNA oligonucleotides (oligomers) supplied by Dharmacon (Lafayette, Colo.) were synthesized to contain free thiols that do not react efficiently with maleimides as supplied, presumably due to disulfide bond formation.

Reduction of thiol modified RNA and DNA oligonucleotides: Thiol-oligomers were reduced using either Dithiothreitol (DTT) immobilized on polyacrylamide beads (REDUCTACRYL®, Calbiochem, San Diego, Calif.), or TCEP (Tris(2-carboxyethyl)phosphine hydrochloride, PierceBio, Rockford, Ill.). After reduction, concentrations of the reduced oligomers were determined using a UV spectrophotomenter. Stored at −20° C., reduced oligomers remained stable and reactive for only two-three weeks.

Chip fabrication: The method of Brockman et al (J. Am. Chem. Soc. 1999 121, 8044-8051) was followed. Briefly, SPOTREADY™ chips were incubated overnight in AOT solution to generate a free amine-activated surface. Addition of SSMCC (sulfosuccinimidyl 4-(N-maleimido-methyl)-cyclohexane-1-carboxylate, PierceBio) then generates a maleimide-activated surface that forms a covalent bond with the thiol group of the oligonucleotides. To quench un-reacted AOT amino groups and thereby minimize nonspecific binding, surfaces were then blocked with PEG-NHS (polyethylene glycol N-hydroxysuccinimide, Nektar Therapeutics Ala., Huntsville, Ala.). At this stage the arrays are stable for several weeks dried or in buffer.

Arrays fabricated using thiolated and biotinylated oligonucleotides were evaluated. The performance of the arrays in hybridizing to DNA is similar for both thiolated or biotinylated oligonucleotides, since the increase in reflectivity upon binding 500 nM complementary tag to either array is indistinguishable (FIG. 10).

### Example 12

### Development of Conditions to Analyze the Levels of Specific mRNAs in a Complex Pool of mRNAs

Conditions for On-Chip RNaseH-Mediated Oligonucleotide Hydrolysis.

Arrays were fabricated using biotinylated oligonucleotides and PEG as negative control. After collection of a reference image, the array was exposed sequentially to complementary tags. FIG. 11 panels B and C illustrates assay specificity: each DNA tag hybridizes only to its complementary DNA and RNA oligonucleotides.

After tag hybridization, the array was exposed to RNaseH: reflectivity decreased only for the RNA oligonucleotides confirming that the RNaseH degrades only RNA-DNA hybrids and not control DNA-DNA hybrids or PEG, as expected under these assay conditions (FIG. 11, panel D). Addition of an excess of complementary DNA tag results in complete removal of RNA oligonucleotide from the surface.

Addition of each DNA tag resulted in a signal change of approx +6 piu (pixel intensity units, the raw SPR response prior to conversion to absolute reflectivity, Δ% R) for the corresponding oligonucleotides prior to RNaseH addition. Following addition of RNaseH, signal change for these oligonucleotide regions is approx −12 piu, i.e. double the amplitude of the DNA binding signal. This is the expected result if both the annealed complement and the immobilized oligonucleotides are fully removed from the modified gold surface. Thus, this assay system works effectively using biotinylated RNA oligonucleotide arrays.

RNase H Reaction Kinetics

The kinetics of RNase H activity on RNA oligonucleotide arrays have been thoroughly studied (Fang et al, Anal. Chem. 2005 77, 6528-6534). Once the enzyme is bound to the substrate, the RNA hydrolysis reaction is very fast (k_{cat}=1 sec⁻¹), such that the increased mass on the array surface due to enzyme and tag DNA binding to RNA oligonucleotides is not significant, and no increase in SPR reflectivity is detectable. Thus binding of target DNA and enzyme to the surface oligonucleotides under assay conditions used for gene expression analysis does not measurably increase reflectivity, and thus not materially impact the measured loss of reflectivity due to oligonucleotides degradation in measurements.

The rate of reaction (loss of surface oligonucleotides) is proportional to the surface coverage of RNA-DNA-enzyme complex, and thus is generally linearly related to DNA tag concentration when tag concentration is low (Fang et al, supra). At higher tag concentrations, the relationship is no longer linear, but the kinetics are still predictable and in theory measured reaction rates can be used to calculate tag concentration.

RNase H Concentration:

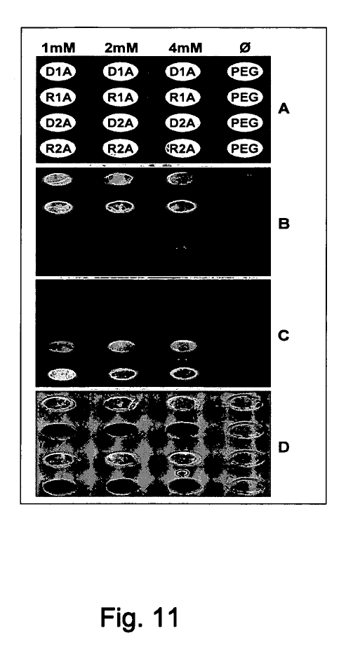

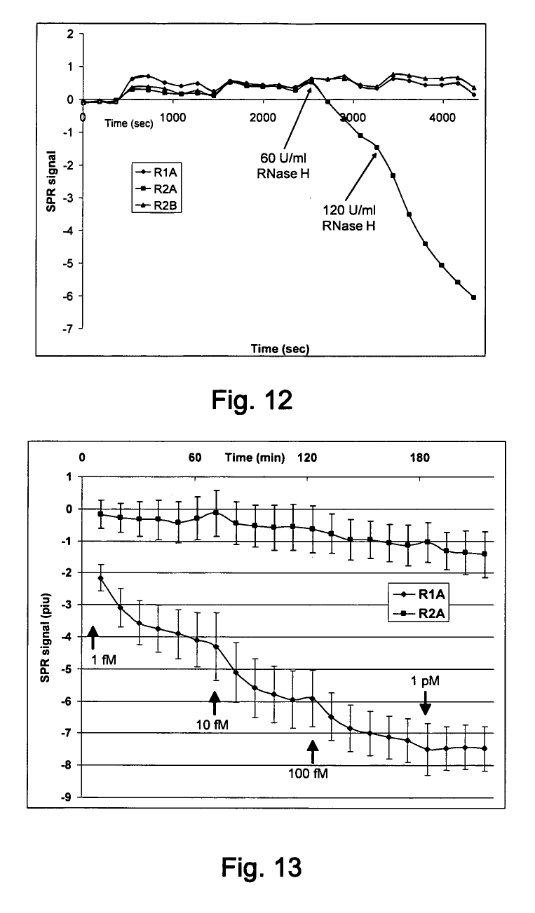

The rate of RNA oligonucleotide degradation is dependent on RNaseH concentration under the current assay conditions (FIG. 12). To allow for high abundance mRNAs, the enzyme should ideally be present at the highest possible concentration in order to ensure efficient degradation. Based on experiments, using a standard stock product (Takara Mirus Bio, Madison, Wis.), maximum practical concentration of RNaseH was found to be about 120 U/mL, although a greater concentration could be readily employed. As shown in FIG. 12, this concentration results in a faster rate of degradation than does 60 U/mL, so the enzyme is not saturating at that concentration. Higher enzyme concentrations could be obtained by using RNaseH at a higher specific activity.

### Example 13

### Using the Method to Detect DNA Tags

The effects of the DNA tag concentration on SPR signal change in the presence of 60 U/mL enzyme at 30° C. were evaluated. In the absence of carrier such as the mRNA that would be present in the gene expression assay, and without significant efforts to optimize conditions, 1 fM of the DNA oligonucleotide was readily detectable (FIG. 13), generating a normalized shift of ˜3 piu greater than the RNA control oligonucleotides. Since, under the conditions used in this assay, the minimum detectable shift with averaging is ˜0.5 piu (corresponding to a reflectivity change of ˜0.13%), one can extrapolate that ˜200 aM (200×10⁻¹⁸ M) tag could be detected, or approximately 100 zeptomoles in the 0.6 mL of sample used in these experiments.

Assuming there are 360,000 mRNAs per mammalian cell and using 1 μg of mRNA per assay, an mRNA molecule present at one copy per cell would be present at a concentration of approximately 18 fM under these assay conditions, i.e. 18× the level of tag detected in this experiment. Thus very rare targets can be detected with this method.

By way of comparison with existing methods, the lower limit of target detection reported using the other array platforms has been reported to be 250 fM for 24 mer oligonucleotides and 50 M for 60 mer oligonucleotides. In certain embodiments, therefore, the instant detection method is potentially >50× more sensitive than other array-based systems for gene expression analysis.

Array Stability Under Assay Conditions

All steps involving RNA prior to exposure to RNase H were performed under conditions designed to avoid and/or inactivate RNases; all buffers were treated with DEPC and were autoclaved. As shown by the experiments so far, RNA oligonucleotides proved very stable under the instant assay conditions. To test longer time frames, the arrays in one experiment were incubated for a total of 6 hours at 30° C. with added RNase H at 120 Units/mL. In the absence of complementary DNA tags, no degradation of RNA control oligonucleotides was observed.

### Example 14

### Conditions for Specific Annealing of DNA Tags to mRNA and for Removal of Free Tags

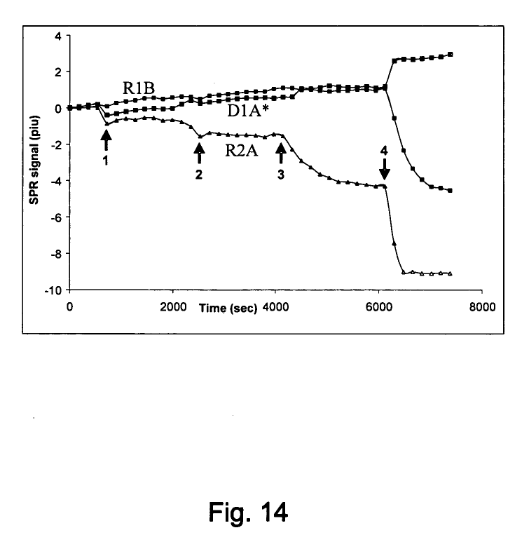

Annealing was done by mixing known concentrations of luc and/or kan mRNA with all tags at a 2× molar excess, heating to 95° C. to denature, and then cooling to 45-50° C. before separation. Stringency was controlled by varying the experimental conditions such as the salt concentration in the annealing buffer (100-300mM) and the temperature to which the mixture was cooled after denaturing (55° C. to room temperature). The molar excess of the DNA tags added to the mRNA was also varied (see below). At the low end (2×) there may be a significant number of target mRNAs with no tags annealed to them but no nonspecific tag carryover is observed (FIG. 14).

Since this assay proved so sensitive for pure tags, it was concluded that the best method to monitor carryover of nonspecific DNA tags. luc mRNA, but not kan mRNA, was mixed with all four DNA tags and 1 μg mouse mRNA as carrier. After annealing, the mixture was put through different separation protocols. Post separation, the purified mRNAs with tags annealed were mixed with RNaseH, diluted to 600 μL, and exposed to an RNA oligonucleotide array. Degradation of luc oligonucleotides would confirm recovery of luc tags coming through the separation step annealed to the luc mRNA; degradation of kan oligonucleotides would indicate carryover of kan tags due to poor separation (FIG. 14).

Separation methods: Several commercially available columns designed for removal of short primers from PCR reaction mixtures were tested, including the QIAquick and the MinElute PCR purification kits from Qiagen Sciences (Germantown, Md.) and the RNeasy MinElute Clean Up kit (Qiagen), which is designed for RNA purification. These columns use guanidinium-HCl (GuHCl).

Gel filtration using the S-300 MicroSpin columns (Amersham, Piscataway, N.J.)) removed free tags very effectively, without the use of the chaotropic salt (FIG. 14). There is no detectable tag carryover (i.e. no significant RNA oligonucleotide degradation) when complementary mRNA is absent (FIG. 14, sample 1). Loss of oligonucleotide was observed when mRNAs were included with the tags, though this loss was less than expected had the tag separation method recovered 100% of the target mRNA with each molecule carrying the annealed DNA tag. For example, loss of reflectivity in sample 3 (FIG. 14) reflects a DNA tag concentration of approximately 250 pM at 100% mRNA+tag recovery; instead the signal loss correlates with the loss observed for about 1-10 fM tag DNA in the purified tag assay (FIG. 13). Most important, however, the removal of non-complementary tags, as evidenced by the lack of degradation of control kan oligonucleotides in this experiment, shows that this method of detecting specific mRNAs in a complex mixture works.

### Example 15

### Improved Sensitivity of Detecting mRNA

To drive hybridization of DNA tags to mRNA molecules, molar excess of DNA tags was increased to 300× versus the 2× excess used above.

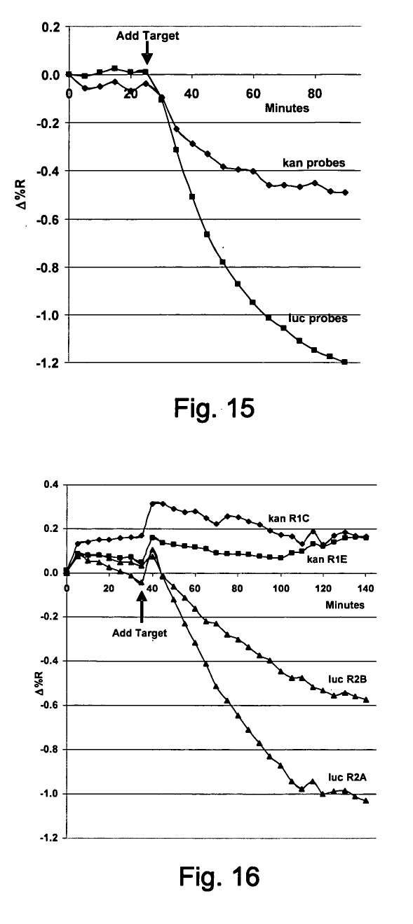

1 μg mouse mRNA was mixed with 5 ng luciferase mRNA (corresponding to ˜0.5% of total mRNA) but no kan mRNA, then mixed with 50 nM of each DNA tag for kanamycin and luciferase mRNAs (˜300× molar tag excess) in 500 mM KCl. The mixture was heated to 95°, gradually cooled to 45°, then chilled and passed over an Amersham MicroSpin S-300 gel filtration column. The mixture was diluted to 600 μL, RNase H was added, and the sample was exposed to an array with kan and luc oligonucleotides. SPR responses were monitored on the SPRimager®II in real time and converted to reflectivity changes (Δ% R) as usual. A significant loss of the luc oligonucleotide from the array was observed (FIG. 15), consistent with detection of luc mRNA at a relative abundance of 0.5%.

The results indicate that reducing the annealing stringency can lead to improved sensitivity, in this case detecting target mRNA at 0.5% abundance. Compared to the detection of 20% mRNA abundance (the detection level achieved in the experimental results shown in FIG. 14) this simple modification to the tag annealing and separation procedure resulted in a ˜40× improvement in sensitivity.

With the 300-fold excess of DNA tags used in this experiment, a weak but significant loss of the control kan oligonucleotides was observed, consistent with some carryover of DNA tag complementary to the control kan mRNA, which was not present in the sample (FIG. 15).

### Example 16

### Application of the AmpliFast™ Method to cDNA Targets

Instead of hybridizing DNA tags to mRNA and specifically recovering hybridized tags for analysis, the mRNA spiked with luc but not kan mRNA was converted into cDNA, which was then exposed directly to RNA oligonucleotide arrays in the presence of RNaseH.

500 ng mouse liver mRNA was mixed with 250 pg luciferase mRNA (corresponding to ˜0.05% of total mRNA). Reverse transcription was primed using equal parts oligo-dT₁₈ primer and random decamer primers with ArrayScript™ reverse transcriptase enzyme (Ambion, Austin, Tex.). This step creates target DNA sequences complementary to the mRNA by extending the oligo-dT or random decamer primers, resulting in extended DNA sequences that are now specific for the complementary mRNA (i.e., DNA probes). After a 2 hour incubation at 42°, the mixture was heated to inactivate the enzyme and denature the cDNA, then kept on ice. The sample was diluted to 600 μL, RNaseH was added and the mixture exposed to an RNA array with 24-mer oligonucleotides specific for luciferase mRNA (R2A and R2B) and for kanamycin (controls R1C, R1D and R1E). Loss of oligonucleotide was monitored on the SPRIMAGER®II. SPR signals from replicate oligonucleotides were averaged and normalized to the RID control, then converted to reflectivity changes, Δ% R (FIG. 16). Loss of signal was observed for both luciferase oligonucleotides, whereas no loss of signal was seen for the kanamycin controls.

These results show that the instant method can be used to measure specific mRNA levels in complex mixtures for targets that are first converted to cDNA. Even before optimization, the method can detect targets present at a relative abundance of less than 0.05%, which corresponds to ˜180 mRNA molecules/mammalian cell.

The specific detection of the luc target in this experiment was observed despite the presence, in the sample exposed to the oligonucleotides array, of random decamers used to prime reverse transcription. Evidently there is no need to separate these primers from the cDNA mixture prior to exposure to the oligonucleotide array.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of sample analysis, comprising:
a) contacting an RNA sample with a DNA oligonucleotide under conditions suitable for hybridization of the DNA oligonucleotide to an RNA molecule in said RNA sample to form an RNA/DNA hybrid;
b) producing a DNA probe using the DNA oligonucleotide of said RNA/DNA hybrid;
c) contacting said DNA probe with a substrate comprising a surface-immobilized RNA oligonucleotide to produce a surface-immobilized RNA/DNA duplex; and
d) detecting RNAseH-dependent cleavage of said surface-immobilized RNA oligonucleotide in said surface-immobilized RNA/DNA duplex;
wherein said detecting indicates the presence of said RNA molecule in said RNA sample.

2. The method of claim 1, wherein said DNA probe is produced by enzymatically extending said DNA oligonucleotide in said RNA/DNA hybrid to produce a primer extension product.

3. The method of claim 2, wherein said DNA oligonucleotide is a random primer or an oligo-dT primer.

4. The method of claim 1, wherein said DNA probe is produced by separating said DNA oligonucleotide in said RNA/DNA duplex from DNA oligonucleotides that are not in said RNA/DNA duplex.

5. The method of claim 4, wherein said DNA oligonucleotide is a gene specific oligonucleotide.

6. The method of claim 1, wherein said substrate is an array of surface immobilized RNA oligonucleotides.

7. The method of claim 1, wherein said detecting is accomplished by comparing a detectable pattern after said RNAseH-dependent cleavage with a detectable pattern before said RNAseH-dependent cleavage.

8. The method of claim 1, wherein said detecting RNAseH-dependent cleavage is by detecting surface plasmon resonance.

9. The method of claim 1, wherein said surface-bound RNA oligonucleotide comprises an optically detectable label.

10. The method of claim 1, wherein said RNA sample is prepared from a cell.

11. A gene expression assay, comprising:
preparing an RNA sample from a cell in which gene expression is to be analyzed; and
performing the method of claim 1 using said RNA sample to obtain data indicating an expression level of one or more genes expressed in the cell.

12. The gene expression assay of claim 11, wherein said data are qualitative with respect to a level of expression of said one or more genes.

13. The gene expression assay of claim 11, wherein said data are analyzed to provide a comparison of relative expression levels of one or more genes in the cell.

14. The gene expression assay of claim 11, wherein the RNA sample is obtained from a test cell and the data regarding expression of said one or more genes is compared to a expression of said one or more genes in a control cell.

15. The gene expression assay of claim 11, wherein the test cell is a diseased cell and the control cell is a non-diseased cell.

16. The gene expression assay of claim 11, wherein the test cell has been treated with a test agent and the control cell is a cell that has not been treated with the test agent.

17. A kit comprising:
an unlabeled DNA oligonucleotide; and
an RNAseH.

18. The kit of claim 17, further comprising reagents for preparing RNA sample.

19. The kit of claim 17, further comprising a surface-immobilized RNA oligonucleotide.

20. The kit of claim 19, wherein said surface-immobilized RNA oligonucleotide is present on an array of surface-immobilized RNA oligonucleotides.


SEQUENCE LISTING (11 sequences)

SEQ ID NO: 1 - DNA, 24 bp, Artificial Sequence
  synthetic oligonucleotide
ggataaaatgcttgatggtcggaa

SEQ ID NO: 2 - DNA, 24 bp, Artificial Sequence
  synthetic oligonucleotide
atcctctagagtcgccacggttga

SEQ ID NO: 3 - DNA, 24 bp, Artificial Sequence
  synthetic oligonucleotide
ctctccagcggttccatcctctag

SEQ ID NO: 4 - DNA, 24 bp, Artificial Sequence
  synthetic oligonucleotide
ttttcgcggttgttacttgactgg

SEQ ID NO: 5 - RNA, 24 nt, Artificial Sequence
  synthetic oligonucleotide
ggaugggaauacucaaccgaugga

SEQ ID NO: 6 - RNA, 32 nt, Artificial Sequence
  synthetic oligonucleotide
aaaaaaaauuccgaccaucaagcauuuuaucc

SEQ ID NO: 7 - RNA, 32 nt, Artificial Sequence; identical to SEQ ID NO: 6
  synthetic oligonucleotide

SEQ ID NO: 8 - DNA, 32 bp, Artificial Sequence
  synthetic oligonucleotide
aaaaaaaattccgaccatcaagcattttatcc

SEQ ID NO: 9 - RNA, 32 nt, Artificial Sequence
  synthetic oligonucleotide
aaaaaaaacuagaggauggaaccgcuggagag

SEQ ID NO: 10 - RNA, 32 nt, Artificial Sequence
  synthetic oligonucleotide
aaaaaaaaccagucaaguaacaaccgcgaaaa

SEQ ID NO: 11 - RNA, 32 nt, Artificial Sequence
  synthetic oligonucleotide
aaaaaaaauccaucgguugaguauucccaucc